(19) United States
(12) Patent Application Publication
(10) Pub. No.: US 2020/0364855 A1
(43) Pub. Date: Nov. 19, 2020

(54) SYSTEM, METHOD AND COMPUTER-ACCESSIBLE MEDIUM FOR CLASSIFYING BREAST TISSUE USING A CONVOLUTIONAL NEURAL NETWORK

(71) Applicant: THE TRUSTEES OF COLUMBIA UNIVERSITY IN THE CITY OF NEW YORK, NEW YORK, NY (US)

(72) Inventor: RICHARD HA, White Plains, NY (US)

(21) Appl. No.: 16/766,123

(22) PCT Filed: Nov. 21, 2018
(86) PCT No.: PCT/US2018/062314

§ 371 Date: May 21, 2020

Related U.S. Application Data
(60) Provisional application No. 62/589,924, filed on Nov. 22, 2017.

(51) Int. Cl. G06T 7/00; G06K 9/62; G06T 11/00; G06T 7/10; G06T 3/40; G06N 3/08; G06N 3/04; G16H 50/20; G16H 30/40; A61B 5/00

(52) U.S. Cl. CPC G06T 7/0012; A61B 2576/02; G06K 9/6279; G06T 11/003; G06T 7/10; G06T 3/4046; G06N 3/08; G06N 3/04; G16H 50/20; G16H 30/40; A61B 5/7264; A61B 5/0066; A61B 5/0091; G06T 2207/20084; G06T 2207/30068; G06T 2207/10101; G06T 2207/30096; G06K 9/6217

(57) ABSTRACT

An exemplary system, method and computer-accessible medium for classifying a breast tissue(s) a patient(s) can include, for example, receiving an image(s) of an internal portion(s) of a breast of the patient(s), and automatically classifying the breast tissue(s) of the breast by applying a neural network(s) to the image(s). The automatic classification can include a classification as to whether the breast tissue(s) is atypical ductal hyperplasia or ductal carcinoma. The automatic classification can include a classification as to whether the breast tissue(s) is a cancerous tissue or a non-cancerous tissue. The image(s) can be a mammographic image or an optical coherence tomography image.

### CROSS-REFERENCE TO RELATED APPLICATION(S)

This application relates to and claims priority from U.S. Patent Application No. 62/589,924, filed on Nov. 22, 2017, the entire disclosure of which is incorporated herein by reference.

### FIELD OF THE DISCLOSURE

The present disclosure relates generally to a classification of information regarding breasts and breast tissue, and more specifically, to exemplary embodiments of systems, methods and computer-accessible medium for classifying breast tissue using a convolutional neural network.

### BACKGROUND INFORMATION

Atypical ductal hyperplasia (“ADH”) is a proliferative epithelial lesion involving the terminal ductal lobular units of the breast that is a non-obligate precursor to invasive disease. ADH is diagnosed by biopsy in up to 15% of suspicious screen-detected lesions, and is often difficult to distinguish from ductal carcinoma in situ (“DCIS”). (See, e.g., Reference 1). While ADH is morphologically very similar to low-grade DCIS, the two entities can be distinguished mainly by quantitative criteria according to the WHO classification. (See, e.g., Reference 2). ADH is limited to a size of ≤2 mm and involves no more than two membrane-bound spaces. Given that the distinction of ADH from DCIS relies on the quantity of atypia present, ADH can often be underestimated by tissue biopsy sampling alone.

Retrospective studies report upgrade rates of ADH to DCIS or invasive cancer of 10-30% at the time of subsequent excision, therefore surgical excision has been the standard of care after a biopsy diagnosis of ADH. (See, e.g., Reference 3). The majority of women with the diagnosis of ADH after biopsy does not upgrade at the time of excision, and therefore undergo the unnecessary morbidity of surgery. Multiple groups have attempted to identify a favorable subset of low-risk patients who can be observed based on various clinical, histologic and/or radiographic criteria. (See, e.g., References 1 and 4-8). In addition, even with the use of vacuum-assisted biopsy procedures, biopsy alone has resulted in unacceptably high rates of upgrade. (See, e.g., Reference 9). To date, these small, retrospective studies have not changed current recommendations.

Mammography is a common imaging modality used in the screening and detection of breast cancers. Little research has been done to evaluate the use of mammography in the detection of intra-tumor heterogeneity (see, e.g., Reference 10), however in recent years there has been growing interest in radiomics. Incorporating advances in machine learning, specifically artificial neural networks called convolutional neural networks (“CNNs”), computational models can extract deep, abstract features of image data sets from hidden layers of CNNs to perform sophisticated classification tasks. (See, e.g., Reference 11).

Women with early-stage breast cancer undergo breast-conserving surgery (“BCS”), which involves the local removal of tumor and surrounding disease-free (e.g., negative) margin. (See, e.g., Reference 25). Approximately 23% of patients may require surgical re-excision (see, e.g., References 26 and 27), which leads to increased healthcare costs and physical and psychological stress on patients and their families. (See, e.g., References 28 and 29). The need for re-excision could be reduced with a rapid intraoperative margin assessment tool.

Optical coherence tomography (“OCT”) is a high-speed, microscopic imaging modality. OCT can be considered as the optical equivalent of ultrasound, relying on the echo of near-infrared light instead of sound to produce micron-scale resolution through 1-2 mm in biological tissue. (See, e.g., Reference 30). In contrast to high-energy X-rays and gamma rays, OCT relies on low-energy near-infrared light, which is non-destructive to tissue, and can resolve microscopic structures that cannot be seen with X-rays or CT, and does not require contrast injection. OCT is an established medical imaging procedure, and has been pioneering in ophthalmology for the past 20 years (see, e.g., References 30-32), promising in cardiology (see, e.g., References 33 and 34), and recently emerging in breast surgery. (See, e.g., References 135-40).

OCT has been investigated as an intraoperative margin assessment technology in breast surgery. This modality has been shown to differentiate normal breast parenchyma such as lactiferous ducts, glands, adipose, and lobules, as well as pathologic conditions such as DCIS, invasive ductal carcinoma (“IDC”), and microcalcifications. (See, e.g., References 41 and 42). OCT facilitates the sample to be studied in the operating room in real-time, which improves the diagnostic speed as compared to histology. OCT has been investigated in a multi-reader clinical study, and it was shown that radiologists can be best-suited for interpreting this modality. (See, e.g., Reference 42). Interpretation of OCT images is typically performed by researchers and clinicians, but manual image interpretation is challenging due to its slow speed and time to train readers, high interobserver variability, and image complexity. As a result, a manual interpretation is not practical in an intraoperative setting. Automated image analysis has the potential to improve diagnostic accuracy with lower interobserver variability and faster speeds, which would increase the clinical impact of OCT and make it more suitable for intraoperative imaging. Deep learning could be used to automatically analyze OCT images.

Deep learning approaches have been developed for OCT imaging and breast cancer imaging, but only a few have investigated using deep learning for OCT imaging of breast cancer. (See, e.g., Reference 43). Deep learning for OCT has been investigated for ophthalmology applications, including quantifying intraretinal fluid (see, e.g., References 44 and 45), and diagnosing retinal disease. (See, e.g., Reference 465). In breast cancer imaging, deep learning has been used to explore several clinical problems using mammograms, MRI scans, and histology. Researchers have investigated classification of breast microcalcifications (see, e.g., Reference 47), and created a breast cancer risk model (see, e.g., Reference 48), using mammographic datasets. Additionally, models have been developed to predict Oncotype Dx recurrence score (see, e.g., Reference 49), post neoadjuvant axillary response (see, e.g., Reference 50), and axillary lymph node metastasis (see, e.g., References 51 and 52) using breast MRI datasets. In pathology, deep learning has been used to detect mitosis in breast cancer histology images. (See, e.g., Reference 53).

Thus, it may be beneficial to provide an exemplary system method and computer-accessible medium for classifying breast tissue using a convolutional neural network which can overcome at least some of the deficiencies described herein above.

### SUMMARY OF EXEMPLARY EMBODIMENTS

An exemplary system, method and computer-accessible medium for classifying a breast tissue(s) a patient(s) can include, for example, receiving an image(s) of an internal portion(s) of a breast of the patient(s), and automatically classifying the breast tissue(s) of the breast by applying a neural network(s) to the image(s). The automatic classification can include a classification as to whether the breast tissue(s) is atypical ductal hyperplasia or ductal carcinoma. The automatic classification can include a classification as to whether the breast tissue(s) is a cancerous tissue or a non-cancerous tissue. The image(s) can be a mammographic image or an optical coherence tomography image.

In some exemplary embodiments of the present disclosure, the neural network can be a convolutional neural network (CNN). The CNN can include a plurality of layers. The layers can include (i) a plurality of residual layers, (ii) a plurality of inception layers, (iii) a fully connected layer(s), and (iv) a linear layer(s). The residual layers can include at least four residual layers, the inception layers can include at least four inception layers, the fully connected layer(s) can include at least sixteen neurons, and the linear layer(s) can include at least eight neurons. The layers can include (i) a plurality of combined convolutional and rectified linear unit (ReLu) layers, (ii) a plurality of partially strided convolutional layers, (iii) a plurality of ReLu layers, and (iv) a plurality of fully connected layer. The combined convolutional and ReLu layers can include at least three combined convolutional and ReLu layers, the partially strided convolutional layers can include at least three partially strided convolutional layers, the ReLu layers can include at least three ReLu layers, and the fully connected layer includes at least 15 fully connected layers.

In certain exemplary embodiments of the present disclosure, a score(s) can be determined based on the image(s) using the neural network(s). The breast tissue can be automatically classified based on the score (e.g., a score above 0.5). The image(s) can illustrate excised breast tissue(s). The image(s) can be segmented and resized prior to classifying the breast tissue. A batch normalization can be performed on the image(s), which can be used so as to limit a drift of layer activations.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIGS. 1A-1C are exemplary atypical ductal hyperplasia input images according to an exemplary embodiment of the present disclosure;

FIGS. 2A-2C are exemplary ductal carcinoma in situ input images according to an exemplary embodiment of the present disclosure;

FIG. 3 is an exemplary flow diagram of a convolutional neural network according to an exemplary embodiment of the present disclosure;

FIG. 4A is an exemplary input magnification mammographic image according to an exemplary embodiment of the present disclosure;

FIG. 4B is an exemplary image of guided backpropagation according to an exemplary embodiment of the present disclosure;

FIG. 4C is an exemplary image with highlighted regions showing positive factor in predicting DCIS according to an exemplary embodiment of the present disclosure;

FIG. 4D is an exemplary attenuation map according to an exemplary embodiment of the present disclosure;

FIG. 5 is an exemplary diagram of OCT image acquisition and labeling according to an exemplary embodiment of the present disclosure;

FIG. 6 is a further exemplary schematic diagram of a further convolutional neural network according to an exemplary embodiment of the present disclosure;

FIG. 7A is an exemplary OCT image of stroma and adipose according to an exemplary embodiment of the present disclosure;

FIG. 7B is an exemplary image of ductal carcinoma in situ according to an exemplary embodiment of the present disclosure;

FIG. 7C is an exemplary image of invasive ductal carcinoma according to an exemplary embodiment of the present disclosure;

FIGS. 7D-7F are exemplary images of the histology corresponding to FIGS. 7A, 7B, and 7C respectively according to an exemplary embodiment of the present disclosure;

FIG. 8A is an exemplary graph of the convergence of Dice coefficients for non-cancer tissue classes according to an exemplary embodiment of the present disclosure;

FIG. 8B is an exemplary graph of the convergence of Dice coefficients for cancer tissue classes according to an exemplary embodiment of the present disclosure;

FIG. 9A is an exemplary OCT image of stroma and adipose according to an exemplary embodiment of the present disclosure;

FIG. 9B is an exemplary image of a duct with adipose tissue according to an exemplary embodiment of the present disclosure;

FIG. 9C is an exemplary image of a terminal ductal lobular unit according to an exemplary embodiment of the present disclosure;

FIGS. 9D-9F are exemplary images of the histology corresponding to FIGS. 9A, 9B, and 9C respectively according to an exemplary embodiment of the present disclosure;

FIG. 10A is an exemplary OCT image of invasive ductal carcinoma according to an exemplary embodiment of the present disclosure;

FIG. 10B is an exemplary image of ductal carcinoma in situ according to an exemplary embodiment of the present disclosure;

FIG. 10C is an exemplary image of a benign cyst with an enlarged duct according to an exemplary embodiment of the present disclosure;

FIGS. 10D-10F are exemplary images of histology corresponding to FIGS. 10A, 10B, and 10C respectively according to an exemplary embodiment of the present disclosure;

FIG. 11 is an even further exemplary diagram of an even further convolutional neural network according to an exemplary embodiment of the present disclosure;

FIG. 12 is an exemplary graph of the convergence of Dice coefficients for cancer tissue classes according to an exemplary embodiment of the present disclosure;

FIG. 13 is an exemplary graph of the convergence of Dice coefficients for non-cancer tissue classes according to an exemplary embodiment of the present disclosure;

FIG. 14 is an exemplary flow diagram of a method for classifying breast tissue of a patient according to an exemplary embodiment of the present disclosure; and

FIG. 15 is an illustration of an exemplary block diagram of an exemplary system in accordance with certain exemplary embodiments of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended claims.

### DETAILED DESCRIPTION OF EXEMPLARY EMBODIMENTS

The exemplary system, method, and computer-accessible medium, according to an exemplary embodiment of the present disclosure, can include the classification of breast tissue (e.g., as a tissue type) using various exemplary imaging modalities. For example, the exemplary system, method, and computer-accessible medium is described below using mammographic images and/or OCT images. However, the exemplary system, method, and computer-accessible medium can also be utilized on other suitable imaging modalities, including, but not limited to, magnetic resonance imaging, positron emission tomography, ultrasound, and computed tomography.

Exemplary Distinguishing Atypical Ductal Hyperplasia from Ductal Carcinoma In Situ

In order to distinguish atypical ductal hyperplasia from ductal carcinoma in situ, two groups were defined. A pure ADH group includes 67 patients who presented with suspicious calcifications without an associated mass on mammogram; had two craniocaudal (“CC”) and mediolateral/lateromedial (“ML/LM”)) magnification views available; and underwent stereotactic guided core biopsy yielding ADH and subsequent surgical excision yielding ADH without upgrade to DCIS. A DCIS group includes 82 patients who presented with suspicious calcifications without an associated mass on mammogram; had two magnification views available; and underwent stereotactic guided core biopsy yielding ADH with subsequent surgical excision yielding upgrade to DCIS (34 patients); underwent stereotactic guided core biopsy yielding ADH and DCIS (21 patients); or stereotactic guided core biopsy yielding DCIS with subsequent surgical excision yielding DCIS without invasion (27 patients).

Clinical pathologic data were collected including age, size and pathology result. Statistical analysis was performed using the IBM SPSS software. Descriptive statistics were used to summarize clinical, imaging, and pathologic parameters. Mammograms were performed on dedicated mammography units (Senographe Essential, GE Healthcare). The views obtained consisted of the standard mediolateral oblique (“MLO”) and CC views. Additional magnification views were obtained of the calcifications in CC and ML/LM projections.

### Exemplary Data Preparation

The ground truth label was extracted from the original pathology report and the data was split into ADH and DCIS groups. Then, the cases were randomly separated into training/validation set, which included 80% of the data, and a test set, which included 20% of the data. The training/validation set was used to develop the exemplary network. The test set, which was set aside prior to training, was used for testing the diagnostic performance of the exemplary procedure.

### Exemplary Data Augmentation and Segregation

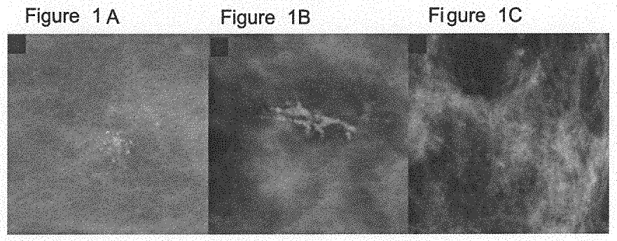

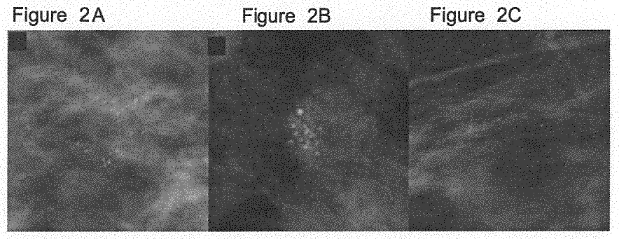

The magnification views of each patient's mammogram were loaded into a 3D segmentation program. Segmentations were manually extracted encompassing the regions of the magnification view which contained calcifications by a fellowship trained breast radiologist with 8 years of experience. Each image was scaled in size based on the radius of the segmentations and resized to fit a 128×128 pixel bounding box. Exemplary atypical ductal hyperplasia input images are shown in FIGS. 1A-1C, and exemplary ductal carcinoma in situ input images are shown in FIG. 2A-2C. The entire image batch was centered by dividing the pixel intensity values by the standard deviation and subtracting by the mean. Data augmentation was performed to limit over-fitting. Images were queued, and were randomly flipped vertically and/or horizontally, rotated by a random angle between +0.52 and −0.52 radians, and randomly cropped to a box 80% of their initial size.

### Exemplary Network Architecture

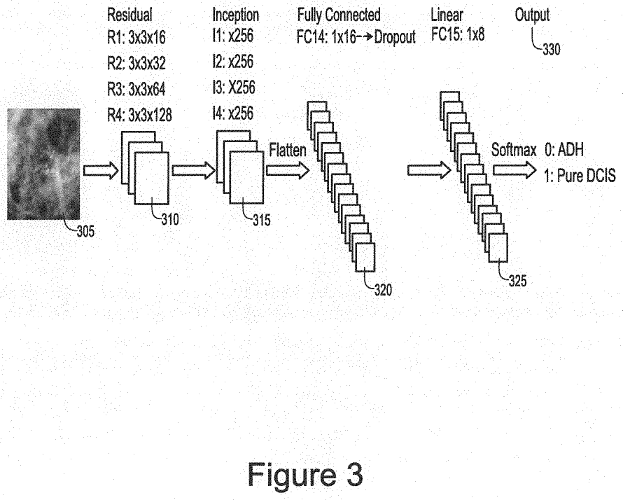

FIG. 3 shows an exemplary flow/schematic diagram of a 15-hidden layer topology used to implement the exemplary neural network according to an exemplary embodiment of the present disclosure. The exemplary fully convolutional neural network can include applying a series of convolution matrices to a vectorized input image 305 that iteratively separates the input to a target vector space. (See, e.g., Reference 12). The network architecture can include 4 residual layers 310 of varying sizes (e.g., (i) 3×3×16, (ii) 3×3×32, (iii) 3×3×64, and (iv) 3×3×128). Residual neural networks can stabilize gradients during back propagation, leading to improved optimization and facilitating greater network depth. Beginning with the 10th hidden layer (e.g., inception layer 315), inception V2 style layers (e.g., ×256 layers) can be utilized. (See, e.g., Reference 13). The Inception layer architecture can be used to implement a computationally efficient method of facilitating a network to selectively determine the appropriate filter architectures to an input feature map, leading to improved learning rates. (See, e.g., Reference 14).

For example, a fully connected layer 320 with 16 neurons can be used after the 13th hidden layer followed by a linear layer 325 with 8 neurons. A final Softmax output layer 330 with two classes was inserted as the last layer. Training was implemented using the Adam optimizer (see, e.g., Reference 15), combined with the Nesterov accelerated gradient. (See, e.g., References 16 and 17). Parameters were initialized using a suitable heuristic. (See, e.g., Reference 18). L2 regularization was implemented to prevent over-fitting of data by limiting the squared magnitude of the kernel weights. Dropout (e.g., 25% randomly) was also employed to prevent over-fitting by limiting unit co-adaptation. (See, e.g., Reference 19). Batch normalization was utilized to improve network training speed and regularization performance by reducing internal covariate shift. (See, e.g., Reference 20).

Softmax with cross entropy hinge loss was utilized as an exemplary objective function of the network to provide a more intuitive output of normalized class probabilities. A class sensitive cost function penalizing incorrect classification of the underrepresented class was utilized. A final softmax score threshold of 0.5 from the average of raw log its from the ML and CC view was used for two class classification. Area under curve (“AUC”) was employed as the performance metric. Sensitivity, specificity and accuracy were also calculated as secondary performance metrics.

Visualization of network predictions was performed using the gradient-weighted class activation mapping (e.g., Grad-CAM). (See, e.g., Reference 21). Each Grad-CAM map was generated by the exemplary prediction model along with every input image. Thus, the salient region of the averaged Grad-CAM map can provide information as to whether these features come from when the prediction model makes classification decisions.

### Exemplary Results

The average age of patients in the ADH group was 55.7 years (SD, 12.9 years). The average age of patients in the DCIS group was 62.1 years (SD, 11.3 years). The differences in age between the two groups was significant (p=0.006). The average size of mammographic calcifications extent of ADH was 1.02 cm (SD, 1.19 cm). The average size of mammographic calcifications extent of DCIS was 1.27 cm (SD, 0.9 cm). The differences in size between the two groups was not significant (p=0.13).

All of the patients underwent stereotactic guided core needle biopsy with a 9 gauge needle. ADH group patients had an average of 9.8 core samples obtained per biopsy (SD 2.5 cores). DCIS group patients had an average of 8.9 core samples obtained per biopsy (SD 2.9 cores). The number of cores between the two groups was not significantly different (p=0.14). DCIS grade was as follows: Low/intermediate grade (48) and high grade (34).

In total, 298 unique images representing ML and CC magnification views of calcifications from 149 patients were used for the exemplary CNN procedure (134 images from 67 patients in the ADH group and 164 images from 82 patients in the DCIS group). The network was trained for 300 epochs. For the test set, the area under the receiver operating curve (e.g., AUC) was 0.86 (95% CI±0.03). Aggregate sensitivity and specificity was 84.6% (95% CI±4%) and 88.2% (95% CI±3%) respectively. Diagnostic accuracy was measured at 86.7% (95% CI, ±2.9).

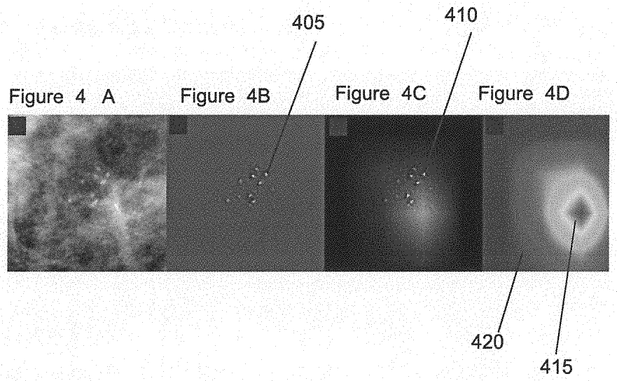

FIGS. 4A-4D illustrate the exemplary generated Grad-CAM maps that indicate salient regions that can include calcifications and the intervening breast parenchyma. An exemplary procedure can be used for visualizing the pixels from an input image (see e.g., input image shown in FIG. 4A) that the network can then evaluate to produce the “guided backpropagation” shown in FIG. 4B. For example, FIG. 4B shows dense and faint calcifications. Every pixel 405 used in making the decision for each class, whether that pixel was a negative or a positive predictor, is highlighted. In GRAD-CAM, as shown in the image of FIG. 4C, areas 410 that are highlighted are the regions that were a positive factor in predicting the specific class. In Guided Grad-CAM, as shown in the map of FIG. 4D, the pixels the network pays attention to are highlighted in order to provide positive inputs which included regions of calcifications as well as intervening breast parenchyma. For example, area 415 indicates the most amount of attention given by the network, and area 420 indicates where the least amount of attention is given by the network. Area 420 also highlights the region of calcifications as well as intervening breast parenchyma.

### Exemplary Discussion

The exemplary results indicate that the exemplary system, method, and computer-accessible medium can distinguish ADH from DCIS using an exemplary CNN, which yielded 86.7% diagnostic accuracy using a mammographic image data set.

Prior groups have identified various clinical, mammographic and/or histologic features to predict for occult malignancy. (See, e.g., References 1, 4, and 7). In a cohort of 140 patients, it was found that that removal of less than 95% of calcifications in the absence of an associated mass, involvement of 2 or more terminal ductal lobular units, the presence of necrosis or significant cytologic atypia, all predicted malignancy. (See, e.g., Reference 1). Using suitable criteria, a cohort of 125 patients with low-risk ADH was selected and observed. (See, e.g., References 1 and 5). At a median follow-up of 3 years, breast cancer events were identified in only 5.6% of the observed group, for example, compared to 12% in a separate intervention group.

In the largest retrospective study conducted over a nine-year period at a single institution of 13,488 consecutive biopsies yielding 422 biopsies with ADH in 415 patients, found that ipsilateral breast symptom, mammographic lesions other than microcalcifications alone, the use of 14 G core-needle biopsy, the presence of severe ADH, co-diagnosis of papilloma and diagnosis of ADH by a pathologist with lower volume independently predicted for malignancy upgrade. They found that even after selection for a low-risk cohort of women, the malignant upgrade frequency at the time of surgery was unacceptably high (17.2% versus 31.3% in all-comers). (See, e.g., Reference 7). Despite large number of studies on this topic, the results can be variable and to date there is no consensus in the selection of low-risk women who can safely undergo observation after a biopsy diagnosis of ADH.

The diagnosis of ADH remains a diagnostic challenge among pathologists, and significant inter-observer variability has been reported. (See, e.g. Reference 11). CNNs have been used in the histopathologic classification of breast biopsy lesions to increase the accuracy and efficiency of diagnosis, and have reported accuracy rates of >80% using relatively small data sets. (See, e.g., References 22 and 23). However, pathology specimen can be limited by the amount of tissue obtained either by core biopsy or surgery.

Other breast imaging modality such as MRI can have a potential role in distinguishing ADH from malignancy. A recent study in 2017 showed patients without suspicious enhancement on breast MRI can be followed rather than undergo surgical excision given the high negative predictive value. (See, e.g., Reference 24). Despite potential for breast MRI for this assessment, typically patients diagnosed with atypia do not undergoes routine breast MRI. As such the study by Tsuchiya only had 17 patients. In addition, the MRI is generally performed after the biopsy and can limit the interpretive value due to post biopsy changes as well as significant removal of the targeted lesion.

In contrast to prior methods, the exemplary system, method, and computer-accessible medium can be used on patients who have mammographic images, which can be prior to the biopsy, to facilitate comprehensive analysis. The exemplary system, method, and computer-accessible medium can utilize a CNN to classify breast cancer lesions based on a mammographic image data set, and further demonstrates the significant potential for radiomics with the utilization of CNNs to change clinical practice. The exemplary system, method, and computer-accessible medium can distinguish ADH from DCIS with 86.7% accuracy using a mammographic dataset. Given the widespread use of screening mammograms, the exemplary system, method, and computer-accessible medium can be used to determine patient management such that patients predicted to have pure ADH lesions can undergo imaging surveillance rather than surgery.

### Exemplary Breast Tissue Classification in Optical Coherence Tomography

### Exemplary Tissue Collection

De-identified human breast tissues from mastectomy and breast reduction specimens were excised from patients. The specimens included both normal and non-neoplastic tissues, and were not needed for diagnosis as defined by the Department of Pathology. The specimens were imaged within 24 hours of surgical excision. Average specimen size was 1.2 cm.²

### Exemplary Imaging Protocol

A custom in-house ultrahigh-resolution OCT (“UHR-OCT”) system centered at 840 nm with an axial resolution of 2.7 μm and lateral resolution of 5.5 μm measured in air was utilized. (See, e.g., Reference 39). The OCT volume included 800 by 800 pixels in the lateral directions covering 3 mm by 3 mm area, and 1024 pixels in the axial direction covering 1.78 mm in depth. All specimens were imaged fresh at room temperature.

### Exemplary Histology

After imaging, tissue specimens were placed in 10% formalin for 24 hours, and then transferred to 70% ethanol for histology processing. Specimen blocks were embedded and sliced along the OCT imaging direction. Multiple 5 μm-thick slices were taken from a single specimen block, with 100 μm discarded between levels, and each slide stained with Hematoxylin and eosin (“H&E”). The processed slides were digitalized at 40× magnification. ImageScope software was used to view and annotate histology images. Histology findings were evaluated by a pathologist with more than 20 years of experience. The dataset of specimens is listed in Table 1 below.

TABLE 1
Distribution of tissue types in dataset. 46 specimens from 23
patients were imaged with a custom UHR-OCT system. 17 specimens
were normal tissue, and 29 specimens were cancer specimens.

| Characteristic | Value (n) |
|---|---|
| Number of patients | 23 |
| Number of specimens | 46 |

Specimen histological confirmations

| | |
|---|---|
| Normal | 17 |
| Cancer | 29 |
| IDC | 24 |
| DCIS | 3 |

### Exemplary Image Labeling

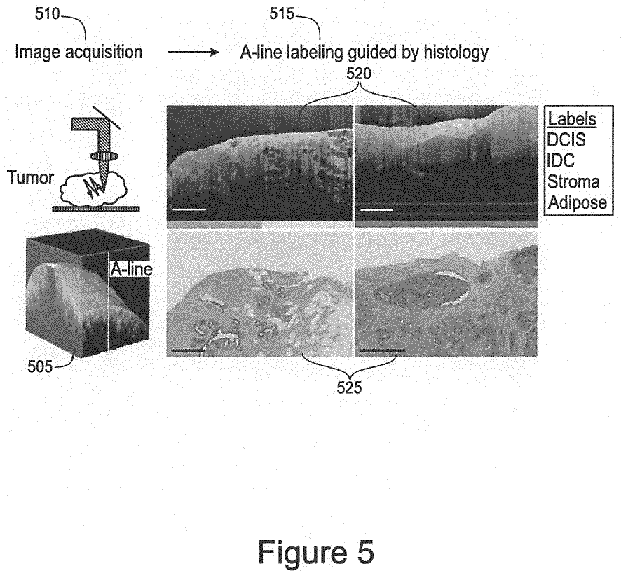

FIG. 5 shows a diagram of exemplary OCT image acquisition and labeling according to an exemplary embodiment of the present disclosure. Each A-line 505 within every OCT B-scan (obtained using image acquisition procedure 510) was manually labeled 515 into four tissue types. Labels were carried out manually, after images 520 were matched with corresponding histology slides 525 and labeled as stroma, adipose, IDC, and DCIS as these are the most common features of breast tissue. The exemplary labeling procedure was carried out using an in-house graphical user interface that facilitates consecutive labeling for three-dimensional data. Two volumes were labeled per specimen per patient for 23 patients, corresponding to 36,800 B-scans. The procedure resulted in 29,440,000 labeled A-lines.

### Exemplary Deep Learning Procedure

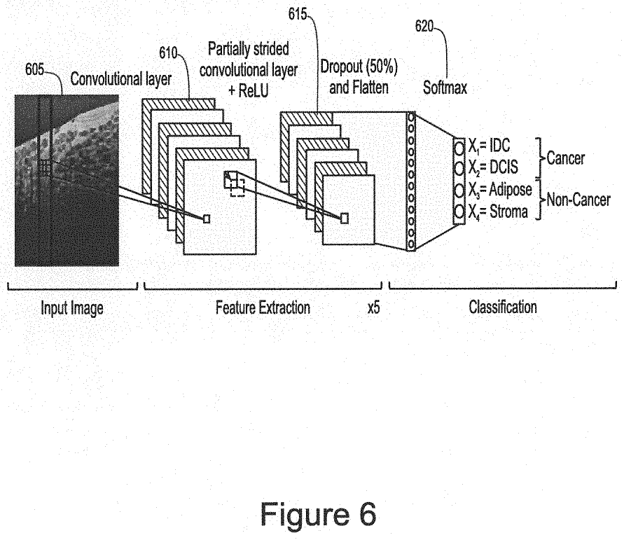

The exemplary deep learning procedure utilized a customized hybrid 2D/1D CNN to map each 2D B-scan to a 1D label vector, which was derived from manual annotation, with a single tissue label class assigned to each A-line in the B-scan. FIG. 6 illustrates a further exemplary schematic diagram of a further convolutional neural network according to an exemplary embodiment of the present disclosure. The exemplary CNN was implemented using an 11-layer architecture including serial 3×3 convolutional filters (see, e.g., Reference 54), with channel sizes increasing from 4 to 64 with increasing convolutional depth (FIG. 6). An image 605 was input into a feature extraction, which included a convolutional layer 610 and a partially strided convolutional layer and rectified linear unit (“ReLu”) 615. Convolutional filters were applied with a stride of 2 in the superficial-to-deep dimension to collapse the image height, while a stride of 1 was applied in the left-to-right dimension to preserve image width. All non-linear functions were modeled by the ReLU. (See, e.g., Reference 55). Batch normalization was performed between the convolutional layer 610 and ReLU layer 615 to limit drift of layer activations during training. (See, e.g., Reference 56). The feature channel sizes increased from 4 to 64 with increasing convolutional depth reflecting increasing representational complexity. A softmax score 620 was generated to determine of the tissue was cancerous or non-cancerous. Annotated images were randomly divided into a training set, which included 80% of the images, and a validation set, which included 20% of the images.

### Exemplary Training

Before training, a two-step pre-processing procedure was used. The original 3-dimensional image volumes were resampled such that each single slice was 256×200 pixels. A simple z-score transformation (x—mean/S.D. of volume) was used to normalize each volume. Second, parameters were initialized using a suitable heuristic. (See, e.g., Reference 57).

Training datasets were generated from the exemplary OCT images with the corresponding labeling. Exemplary training was implemented using an Adam optimizer, a procedure for first-order gradient-based optimization of stochastic objective functions (see, e.g., Reference 58), and standard stochastic gradient descent procedure with Nesterov momentum. (See, e.g., Reference 59). L2 regularization was implemented, e.g., to prevent over-fitting of data by limiting the squared magnitude of the kernel weights. To account for training dynamics, the learning rate was annealed and the mini-batch size was increased whenever training loss plateaus. A normalized gradient procedure was utilized to facilitate locally adaptive learning rates that can adjust according to changes in the input signal. (See, e.g., Reference 60).

### Exemplary Validation and Visualization

The exemplary classification procedure was executed on the validation set and evaluated for accuracy for each tissue type. Given the relatively small number of image volumes, but the relatively large number of B-scans per volume, each volume was divided into multiple 200-slice blocks for training and validation. Five-fold cross-validation was used to estimate accuracy over the entire dataset. Correlation with manual annotations was calculated using a Dice similarity coefficient.

### Exemplary Results

An exemplary manual segmentation of 29,440,000 A-lines from 36,800 OCT B-scans in 46 volumetric datasets were used for training and validation. The annotated images were randomly divided into a training set, which included 80% of the images, and a validation set, which included 20% of the images, and then five-fold cross-validation was performed to ensure that all data was tested in the validation dataset. In each exemplary experiment, 23,552,000 A-lines were used as the training set, and the remaining 5,888,000 A-lines were used for cross-validation. Each B-scan was divided into chunks of 200 A-lines, and each chunk was then randomly divided into training and validation. The procedure was trained over 25,000 iterations, which took about 60 minutes.

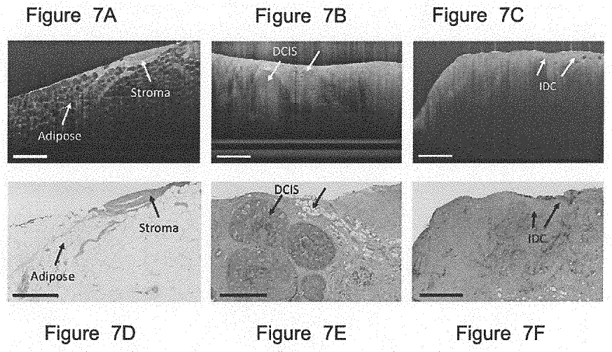

Four different breast tissue structures were classified using the exemplary CNN. Examples of these features of breast tissue in OCT images and corresponding H&E histology are illustrated in the exemplary images shown in FIGS. 7A-7F. For example, FIG. 7A shows an exemplary OCT image of stroma and adipose according to an exemplary embodiment of the present disclosure. FIG. 7B illustrates an exemplary image of ductal carcinoma in situ according to an exemplary embodiment of the present disclosure. FIG. 7C shows an exemplary image of invasive ductal carcinoma according to an exemplary embodiment of the present disclosure. FIGS. 7D, 7E and 7F illustrate exemplary images of histology corresponding to FIGS. 7A, 7B and 7C, respectively, according to an exemplary embodiment of the present disclosure. Healthy breast tissue can be primarily composed of fibrous stroma and adipose, an included the noncancerous tissue class. DCIS, which represents early-stage cancer that has not spread from ducts, and IDC, the most common form of breast cancer that invades surrounding fibrous or adipose tissue, represent the cancerous tissue class. The exemplary procedure was used to classify these four tissue types individually, as well as perform a binary classification (e.g., cancer vs. no-cancer classification).

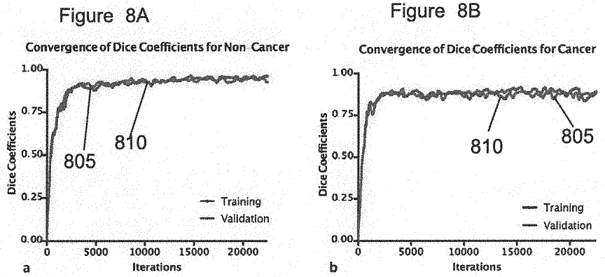

A performance of the exemplary procedure was evaluated using Dice coefficients and the convergence of the procedure was plotted over multiple iterations. The Dice coefficient is a measure of similarity between two samples, and can be commonly used to assess the performance of image segmentation procedures. The exemplary images were manually annotated by the OCT readers, and considered to be the ground truth, and then the exemplary CNN was used the classify the images, and the similarity between the annotations was calculated for the entire dataset. The mean five-fold validation Dice coefficient was highest for IDC (e.g., mean standard deviation, about 0.89±0.09) and adipose (about 0.79±0.17), followed by stroma (about 0.74±0.18), and DCIS (about 0.65±0.15). (See e.g., Table 2 below). IDC and DCIS were combined as single class (e.g., cancer), and adipose and stroma were combined as the non-cancer class, for the case where deep learning can be used to identify images with suspicious areas that need to be investigated further. Using this binary classification, the mean five-fold validation Dice coefficient for cancer was about 0.88±0.04, and about 0.84±0.06 for non-cancer. The convergence of the binary classification is shown in the graphs shown in FIGS. 8A and 8B, which illustrate the plots of the validation set 805 and the training set 810. The procedure converged over 25,000 iterations.

TABLE 2
Distribution of tissue types in dataset and corresponding
five-fold cross-validation Dice scores.

| | | Binary classification |
|---|---|---|
| | Five-fold | five-fold |
| | cross-validation | cross-validation |
| Tissue Type | Dice scores | Dice scores |
| IDC | 0.82-0.95 | 0.84-0.94 |
| DCIS | 0.54-0.75 | |
| Adipose | 0.67-0.91 | 0.81-0.93 |
| Stroma | 0.61-0.86 | |

### Exemplary Discussion

The exemplary system, method, and computer-accessible medium, according to an exemplary embodiment of the present disclosure, can utilize an exemplary CNN that achieved Dice coefficients of 0.89-0.93 in a binary classification of detecting cancerous versus non-cancerous tissue in OCT images of breast specimens. Thus, the exemplary system, method, and computer-accessible medium according to an exemplary embodiment of the present disclosure can be used for deep learning for intraoperative margin assessment of breast cancer and to reduce re-excision rates. IDC and adipose were the easiest to classify, followed by DCIS and stroma. IDC attenuates the OCT strongly, and has an easily recognizable characteristic appearance, and adipose has a distinct honeycomb structure which can also be easier to identify than stroma and DCIS, which have more subtle features.

The exemplary system, method, and computer-accessible medium, according to an exemplary embodiment of the present disclosure, can increase an accuracy when compared to other image classification frameworks developed for detecting breast cancer in OCT images. The exemplary system, method, and computer-accessible medium was able to achieve, using a relevance vector machine to classify IDC and surrounding stroma, an overall accuracy of 84% using data from the same UHR-OCT system. (See, e.g., Reference 39). The binary classification using the exemplary deep learning procedure performed better than traditional image processing procedures. Additionally, classifying OCT images of breast tissue has been investigated in a multi-reader study. (See, e.g., Reference 42). The exemplary CNN had comparable results to the 0.88 accuracy of 7 clinician readers combined, including radiologists, pathologists, and surgeons.

The exemplary system, method, and computer-accessible medium, according to an exemplary embodiment of the present disclosure, can be used as a procedure for improving clinical decision making in the intraoperative setting. Exemplary OCT techniques can differentiate normal breast parenchyma such as lactiferous ducts, glands, adipose, and lobules, as well as pathologic conditions such as DCIS, IDC, and microcalcifications. (See, e.g., Reference 41). In a multi-reader study, clinicians (e.g., radiologists, surgeons, and pathologists) were trained to distinguish suspicious from non-suspicious areas of post-lumpectomy specimens using OCT images, and the results showed that readers from different specialties could accurately read OCT images with relatively short training time (e.g., 3.4 hours). Radiologists achieved the highest accuracy (94%) followed by pathologists and surgeons. All clinical readers had an average accuracy of 88%. These results further validated the feasibility of the exemplary CNN to use OCT as a real-time intraoperative margin assessment tool in BCS. Although clinicians can be trained to read OCT images, there remain practical concerns of high interobserver variability and slow speed, which make manual interpretation impractical for the intraoperative setting.

Thus, the exemplary system, method, and computer-accessible medium can utilize an exemplary CNN to classify cancer in OCT images of breast based on A-line based classification procedures that can be used in real-time applications, and can be extended beyond breast imaging to other applications. Automated processing using the exemplary CNN can overcome challenges of interobserver variability and improve speed in OCT image interpretation. The exemplary CNN facilitates the use of OCT in an intraoperative setting for margin assessment.

### Exemplary OCT-Based Post-Surgical Breast Tumor Specimen Margin Evaluation

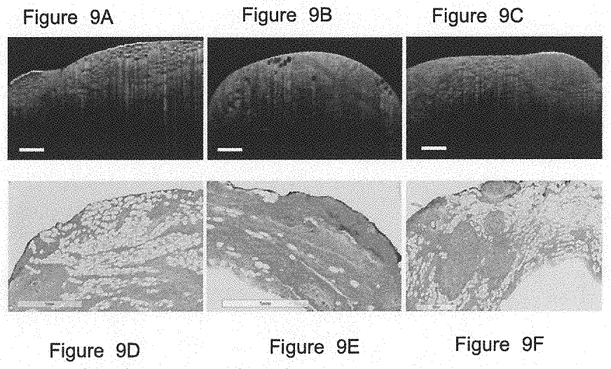

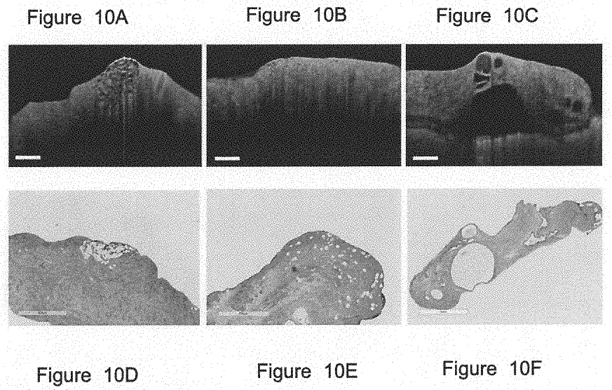

As shown in the exemplary images of FIGS. 9A-9F and 10A-10F, the exemplary system, method, and computer-accessible medium can use ultrahigh-resolution OCT to differentiate relevant structures and the resultant data can be used for automated image analysis. (See, e.g., Reference 80).

### Exemplary Tissue Collection

As indicated in Table 3 below, de-identified normal and non-neoplastic human breast tissues from mastectomy and breast reduction specimens were excised from patients.

TABLE 3
Characteristics of specimens

| Characteristics | Value |
|---|---|
| Number of patients | 49 |
| Number of specimens | 82 |

Specimens imaged by UHR OCT, n (%)

| | | |
|---|---|---|
| IDC | 20 | (38.5) |
| DCIS | 3 | (5.8) |
| Phyllodes | 2 | (3.8) |
| Fibrotic focus carcinoma | 1 | (1.9) |
| Mucinous carcinoma | 3 | (5.8) |
| Normal | 23 | (44.2) |

Specimens imaged by Thorlabs Telesto, n (%)

| | | |
|---|---|---|
| IDC | 7 | (23.3) |
| ILC | 3 | (10.0) |
| DCIS | 3 | (10.0) |
| Normal | 17 | (56.6) |

### Exemplary Imaging Protocol and Histology

Two spectral-domain OCT systems were used for imaging: (i) Thorlabs Telesto I centered at 1300 nm (e.g., axial resolution: 6.5 μm; lateral resolution: 15 μm in air) and (ii) a Custom UHR-OCT system centered at 800 nm (e.g., axial resolution: 2.7 μm; lateral resolution: 5.5 μm in air). Specimens were imaged fresh and submitted for histology. Exemplary histology was evaluated by a pathologist and OCT images were evaluated by authors using corresponding histology.

### Exemplary OCT Image Labeling

Each A-line was labeled for six tissue types: (i) IDC, (ii) DCIS, (iii) mucinous carcinoma, (iv) Phyllodes sarcoma, (v) stroma, (vi) adipose. Labeling procedures used a custom graphical user interface (“GUI”). Two volumes/patient were labeled for 23 patients, resulting in 37 k B-scans and 29.5 million A-lines.

### Exemplary CNN Architecture

The exemplary CNN utilized a hybrid 2D/1D CNN to map each B-scan to a 1D label vector derived from manual annotation. The exemplary CNN was implemented using an exemplary 11-layer architecture consisting of a series of 3×3 convolutional kernels. Non-linear functions modeled by the ReLU. Batch normalization was used between the convolutional and ReLU layers to limit drift of layer activations during training. Feature channel sizes increased from 4 to 64 with increasing convolutional depth reflecting increasing complexity.

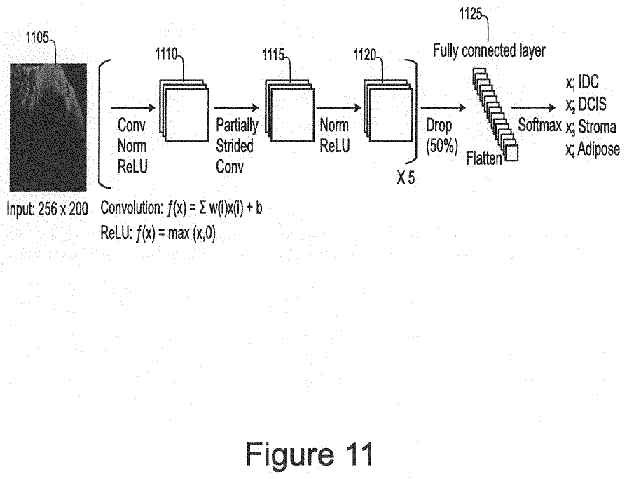

FIG. 11 shows an even further exemplary schematic diagram of an even further convolutional neural network according to an exemplary embodiment of the present disclosure. For example, an image 1105 can be input into a combined convolutional and ReLu layer 1110, which can include three layers. A partially strided convolutional layer 1115, which can include three layers, can feed into a normal ReLu layer 1120, which can include three layers. Multiple fully connected layers 1125, which can include 15 layers, can be used to produce a softmax score, which can be used to differentiate cancerous and non-cancerous tissue.

### Exemplary Training

Annotated exemplary images were randomly divided into a training set, which include 80% of the images, and a validation set, which included 20% of the images. Training was implemented using the Adam optimizer. L2 regularization was implemented to prevent over-fitting of data by limiting the squared magnitude of the kernel weights. To account for training dynamics, the learning rate was annealed and the mini-batch size was increased whenever training loss plateaued. An exemplary normalized gradient procedure was utilized to facilitate locally adaptive learning rates that adjust with changes in input.

### Exemplary Validation and Visualization

The exemplary CNN was performed on the validation set and was evaluated for accuracy for each tissue type. Each volume was divided in 200-slice blocks for training and validation. Five-fold cross-validation was used to estimate accuracy over the entire dataset. Correlation with manual annotations was calculated using a Dice score coefficient:

$$Dice = \frac{2 \vert X ⋂ Y \vert}{\vert X \vert + \vert Y \vert}$$

### Exemplary Results

A total of 30 optical imaging volumes resulting in 26,172 slices were used for preliminary training. For each slice, a total of four tissue types were annotated on a column-by-column basis. The distribution of tissue types was a follows:

| | Distribution of |
|---|---|
| Tissue Type | tissue types |
| IDC | 38% |
| DCIS | 4.4% |
| Adipose | 13% |
| Stroma | 30% |
| N/A | 14% |

Five-fold cross validation yielded Dice scores across the tissue types as follows:

| | Five-fold |
|---|---|
| | cross-validation |
| Tissue Type | Dice score |
| IDC | 0.82-0.95 |
| DCIS | 0.54-0.75 |
| Adipose | 0.67-0.91 |
| Stroma | 0.61-0.86 |

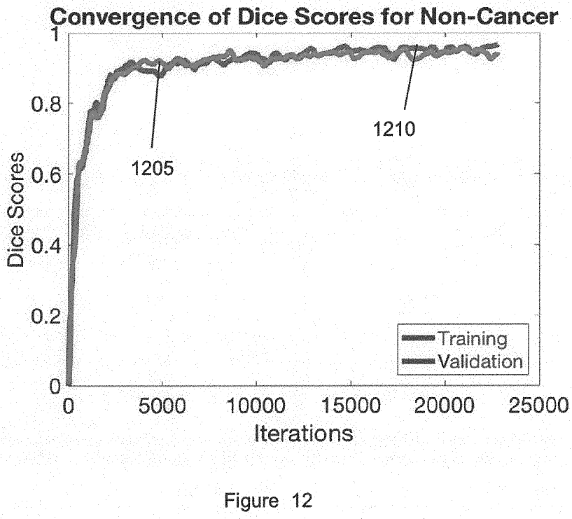

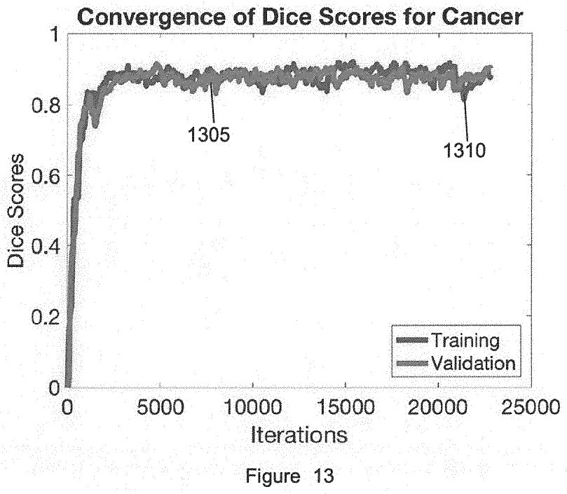

In a second experiment, IDC and DCIS were combined as a single tissue class (e.g., malignancy) while stroma and adipose were combined as a second tissue class (e.g., non-malignancy). In this setup, binary of differentiation of malignancy from non-malignant tissues yielded five-fold cross-validation Dice scores of 0.85-0.92 as shown in the graphs of FIGS. 12 and 13. The convergence of the binary classification illustrates the plots of the validation sets 1205 and 1305, and the training sets 1210 and 1310.

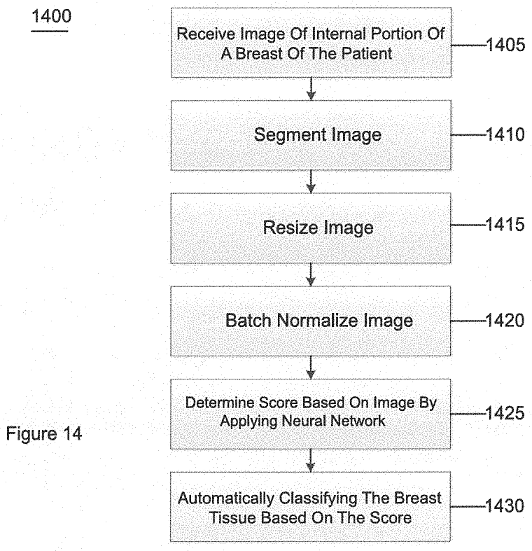

FIG. 14 shows an exemplary flow diagram of a method 1400 for classifying breast tissue of a patient according to an exemplary embodiment of the present disclosure. For example, at procedure 1405, an image of an internal portion of a breast of a patient can be received. At procedure 1410, the image can be segmented, at procedure 1415, the image can be resized, and at procedure 1420, the image can be batch normalized. At procedure 1425, a scored can be determined based on the image by applying a neural network (e.g., the exemplary CNN) to the image. At procedure 1430, the breast tissue can be automatically classified based on the score.

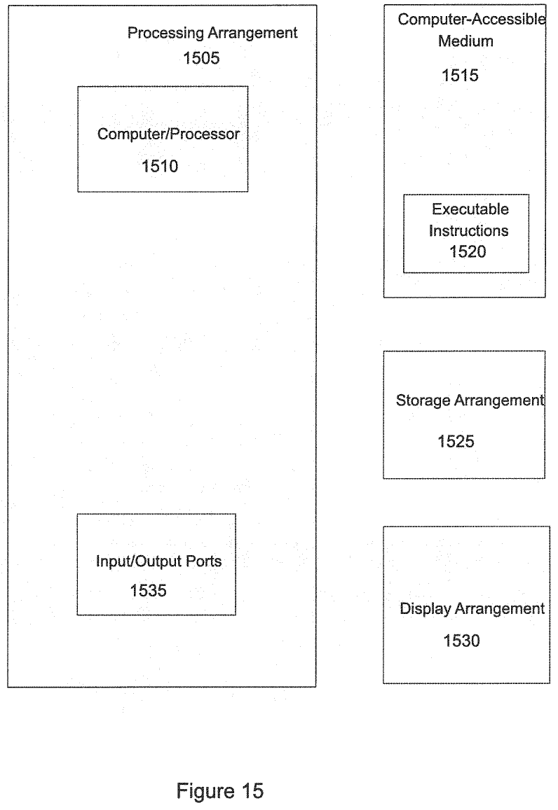

FIG. 15 shows a block diagram of an exemplary embodiment of a system according to the present disclosure. For example, exemplary procedures in accordance with the present disclosure described herein can be performed by a processing arrangement and/or a computing arrangement (e.g., computer hardware arrangement) 1505. Such processing/computing arrangement 1505 can be, for example entirely or a part of, or include, but not limited to, a computer/processor 1510 that can include, for example one or more microprocessors, and use instructions stored on a computer-accessible medium (e.g., RAM, ROM, hard drive, or other storage device).

As shown in FIG. 15, for example a computer-accessible medium 1515 (e.g., as described herein above, a storage device such as a hard disk, floppy disk, memory stick, CD-ROM, RAM, ROM, etc., or a collection thereof) can be provided (e.g., in communication with the processing arrangement 1505). The computer-accessible medium 1515 can contain executable instructions 1520 thereon. In addition or alternatively, a storage arrangement 1525 can be provided separately from the computer-accessible medium 1515, which can provide the instructions to the processing arrangement 1505 so as to configure the processing arrangement to execute certain exemplary procedures, processes, and methods, as described herein above, for example.

Further, the exemplary processing arrangement 1505 can be provided with or include an input/output ports 1535, which can include, for example a wired network, a wireless network, the internet, an intranet, a data collection probe, a sensor, etc. As shown in FIG. 15, the exemplary processing arrangement 1505 can be in communication with an exemplary display arrangement 1530, which, according to certain exemplary embodiments of the present disclosure, can be a touch-screen configured for inputting information to the processing arrangement in addition to outputting information from the processing arrangement, for example. Further, the exemplary display arrangement 1530 and/or a storage arrangement 1525 can be used to display and/or store data in a user-accessible format and/or user-readable format.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

### EXEMPLARY REFERENCES

The following references are hereby incorporated by reference in their entireties:
1. Nguyen C V, Albarracin C T, Whitman G J, et al. Atypical ductal hyperplasia in directional vacuum-assisted biopsy of breast microcalcifications: considerations for surgical excision. Ann Surg Oncol 18:752-61, 2011.
2. Sinn H P, Kreipe H. A Brief Overview of the WHO Classification of Breast Tumors, 4th Edition, Focusing on Issues and Updates from the 3rd Edition. Breast Care (Basel) 8:149-54, 2013.
3. Racz J M, Carter J M, Degnim A C. Lobular Neoplasia and Atypical Ductal Hyperplasia on Core Biopsy: Current Surgical Management Recommendations. Ann Surg Oncol, 2017
4. Ko E, Han W, Lee J W, et al. Scoring system for predicting malignancy in patients diagnosed with atypical ductal hyperplasia at ultrasound-guided core needle biopsy. Breast Cancer Res Treat 112:189-95, 2008.
5. Menen R S, Ganesan N, Bevers T, et al. Long-Term Safety of Observation in Selected Women Following Core Biopsy Diagnosis of Atypical Ductal Hyperplasia. Ann Surg Oncol 24:70-76, 2017.
6. Pankratz V S, Hartmann L C, Degnim A C, et al. Assessment of the accuracy of the Gail model in women with atypical hyperplasia. J Clin Oncol 26:5374-9, 2008.
7. Deshaies I, Provencher L, Jacob S, et al. Factors associated with upgrading to malignancy at surgery of atypical ductal hyperplasia diagnosed on core biopsy. Breast 20:50-5, 2011.
8. Bendifallah S, Defert S, Chabbert-Buffet N, et al. Scoring to predict the possibility of upgrades to malignancy in atypical ductal hyperplasia diagnosed by an 11-gauge vacuum-assisted biopsy device: an external validation study. Eur J Cancer 48:30-6, 2012.
9. Yu Y H, Liang C, Yuan X Z. Diagnostic value of vacuum-assisted breast biopsy for breast carcinoma: a meta-analysis and systematic review. Breast Cancer Res Treat 120:469 79, 2010.
10. Song J L, Chen C, Yuan J P, et al. Progress in the clinical detection of heterogeneity in breast cancer. Cancer Med 5:3475-3488, 2016.
11. Gomes D S, Porto S S, Balabram D, et al. Inter-observer variability between general pathologists and a specialist in breast pathology in the diagnosis of lobular neoplasia, columnar cell lesions, atypical ductal hyperplasia and ductal carcinoma in situ of the breast. Diagn Pathol 9:121, 2014.
12. LeCun, Yann, et al. “Gradient-based learning applied to document recognition.” Proceedings of the IEEE 86.11 (1998): 2278-2324.
13. He, Kaiming, et al. “Deep residual learning for image recognition.” Proceedings of the IEEE conference on computer vision and pattern recognition. 2016.
14. Szegedy, Christian, et al. “Going deeper with convolutions.” Proceedings of the IEEE conference on computer vision and pattern recognition. 2015.
15. Kingma, D P, B A J. “Adam: A method for stochastic optimization.” arXiv preprint arXiv:1412.6980 (2014).
16. Nesterov, Yurii. “Gradient methods for minimizing composite objective function.” (2007).
17. Dozat, Timothy. “Incorporating nesterov momentum into adam.” (2016).
18. Glorot, Xavier, and Yoshua Bengio. “Understanding the difficulty of training deep feedforward neural networks.” Proceedings of the Thirteenth International Conference on Artificial Intelligence and Statistics. 2010.
19. Srivastava N, Hinton G E, Krizhevsky A, et al. “Dropout: a simple way to prevent neural networks from overfitting.” Journal of machine learning research 15.1 (2014): 1929-1958.
20. Ioffe, Sergey, and Christian Szegedy. “Batch normalization: Accelerating deep network training by reducing internal covariate shift.” International Conference on Machine Learning. 2015.
21. Ramprasaath R S, Abhishek D, Ramakrishna V, et al. 2016 Grad-CAM: why did you say that? visual explanations from deep networks via gradient-based localization CVPR 2016 (arXiv:1610.02391)
22. Araujo T, Aresta G, Castro E, et al. Classification of breast cancer histology images using Convolutional Neural Networks. PLoS One 12:e0177544, 2017
23. Bejnordi B E, Zuidhof G, Balkenhol M, et al. Context-aware stacked convolutional neural networks for classification of breast carcinomas in whole-slide histopathology images. J Med Imaging (Bellingham) 4:044504, 2017
24. Tsuchiya K, Mori N, Schacht D, et al. Value of breast MRI for patients with a biopsy showing atypical ductal hyperplasia (ADH). J Magn Reson Imaging. 2017 December; 46(6):1738-1747.
25. K. B. Clough, J. S. Lewis, B. Couturaud, A. Fitoussi, C. Nos, and M. C. Falcou, “Oncoplastic techniques allow extensive resections for breast-conserving therapy of breast carcinomas,” Annals Surg. 237, 26-34 (2003).
26. P. I. Tartter, J. Kaplan, I. Bleiweiss, C. Gajdos, A. Kong, S. Ahmed, and D. Zapetti, “Lumpectomy margins, reexcision, and local recurrence of breast cancer,” The Am. J. Surg. 179, 81-85 (2000).
27. L. E. McCahill, R. M. Single, E. J. A. Bowles, H. S. Feigelson, T. A. James, T. Barney, J. M. Engel, and A. A. Onitilo, “Variability in Reexcision Following Breast Conservation Surgery,” JAMA: The J. Am. Med. Assoc. 307, 467-475 (2012).
28. J. F. Waljee, E. S. Hu, L. A. Newman, and A. K. Alderman, “Predictors of re-excision among women undergoing breast-conserving surgery for cancer,” Annals Surg. Oncol. 15, 1297-1303 (2008).
29. M. A. Olsen, K. B. Nickel, J. A. Margenthaler, A. E. Wallace, D. Mines, J. P. Miller, V. J. Fraser, and D. K. Warren, “Increased Risk of Surgical Site Infection Among Breast-Conserving Surgery Re-excisions,” Annals Surg. Oncol. 22, 2003-2009 (2015).
30. D. Huang, E. A. Swanson, C. P. Lin, J. S. Schuman, W. G. Stinson, W. Chang, M. R. Hee, T. Flotte, K. Gregory, C. A. Puliafito, A. Et, and et al., “Optical coherence tomography.” Science 254, 1178-81 (1991).
31. M. Adhi and J. S. Duker, “Optical coherence tomography-current and future applications,” Curr. Opin. Ophthalmol. 24, 213-221 (2013).
32. C. A. Puliafito, M. R. Hee, C. P. Lin, E. Reichel, J. S. Schuman, J. S. Duker, J. A. Izatt, E. A. Swanson, and J. G. Fujimoto, “Imaging of Macular Diseases with Optical Coherence Tomography,” Ophthalmology 102, 217-229 (1995).
33. I.-K. Jang, B. E. Bouma, D.-H. Kang, S.-J. Park, S.-W. Park, K.-B. Seung, K.-B. Choi, M. Shishkov, K. Schlendorf, E. Pomerantsev, S. L. Houser, H. Aretz, and G. J. Tearney, “Visualization of coronary atherosclerotic plaques in patients using optical coherence tomography: comparison with intravascular ultrasound,” J. Am. Coll. Cardiol. 39, 604-609 (2002).
34. T. Kubo, T. Imanishi, S. Takarada, A. Kuroi, S. Ueno, T. Yamano, T. Tanimoto, Y. Matsuo, T. Masho, H. Kitabata, K. Tsuda, Y. Tomobuchi, and T. Akasaka, “Assessment of Culprit Lesion Morphology in Acute Myocardial Infarction,” J. Am. Coll. Cardiol. 50, 933-939 (2007).
35. W. Luo, F. T. Nguyen, A. M. Zysk, T. S. Ralston, J. Brockenbrough, D. L. Marks, A. L. Oldenburg, and S. A. Boppart, “Optical Biopsy of Lymph Node Morphology using Optical Coherence Tomography,” Technol. Cancer Res. & Treat. 4, 539-547 (2005).
36. F. T. Nguyen, A. M. Zysk, E. J. Chaney, J. G. Kotynek, J. Uretz, F. J. Bellafiore, K. M. Rowland, P. A. Johnson, and S. A. Boppart, “Intraoperative Evaluation of Breast Tumor Margins with Optical Coherence Tomography,” Cancer Res. 69, 8790-8796 (2009).
37. K. M. Kennedy, R. A. McLaughlin, B. F. Kennedy, A. Tien, B. Latham, C. M. Saunders, and D. D. Sampson, “Needle optical coherence elastography for the measurement of microscale mechanical contrast deep within human breast tissues,” J. Biomed. Opt. 18, 121510 (2013).
38. L. Scolaro, R. A. McLaughlin, B. F. Kennedy, C. M. Saunders, and D. D. Sampson, “A review of optical coherence tomography in breast cancer,” Photonics & Lasers Medicine 3 (2014).
39. X. Yao, Y. Gan, E. Chang, H. Hibshoosh, S. Feldman, and C. Hendon, “Visualization and tissue classification of human breast cancer images using ultrahigh-resolution OCT,” Lasers Surg. Medicine 49, 258-269 (2017).
40. B. J. Vakoc, D. Fukumura, R. K. Jain, and B. E. Bouma, “Cancer imaging by optical coherence tomography: preclinical progress and clinical potential,” Nat. Rev. Cancer 12, 363 (2012).
41. P. Hsiung, D. R. Phatak, Y. Chen, A. D. Aguirre, J. G. Fujimoto, and J. L. Connolly, “Benign and malignant lesions in the human breast depicted with ultrahigh resolution and three-dimensional optical coherence tomography.” Radiology 244, 865-74 (2007).
42. R. Ha, L. C. Friedlander, H. Hibshoosh, C. Hendon, S. Feldman, S. Ahn, H. Schmidt, M. K. Akens, M. Fitzmaurice, B. C. Wilson, and V. L. Mango, “Optical Coherence Tomography,” Acad. Radiol. 25, 279-287 (2018).
43. A. R. Triki, M. B. Blaschko, Y. M. Jung, S. Song, H. J. Han, S. I. Kim, and C. Joo, “Intraoperative margin assessment of human breast tissue in optical coherence tomography images using deep neural networks,” (2017).
44. C. Lee, A. Tyring, N. Deruyter, Y. Wu, A. Rokem, and A. Lee, “Deep-learning based, automated segmentation of macular edema in optical coherence tomography,” Biomed. Opt. Express 8 (2017).
45. F. G. Venhuizen, B. van Ginneken, B. Liefers, F. van Asten, V. Schreur, S. Fauser, C. Hoyng, T. Theelen, and C. I. Sanchez, “Deep learning approach for the detection and quantification of intraretinal cystoid fluid in multivendor optical coherence tomography,” Biomed. Opt. Express 9, 1545-1569 (2018).
46. J. De Fauw, J. R. Ledsam, B. Romera-Paredes, S. Nikolov, N. Tomasev, S. Blackwell, H. Askham, X. Glorot, B. OâAZDonoghue, D. Visentin, G. van den Driessche, B. Lakshminarayanan, C. Meyer, F. Mackinder, S. Bouton, K. Ayoub, R. Chopra, D. King, A. Karthikesalingam, C. O. Hughes, R. Raine, J. Hughes, D. A. Sim, C. Egan, A. Tufail, H. Montgomery, D. Hassabis, G. Rees, T. Back, P. T. Khaw, M. Suleyman, J. Cornebise, P. A. Keane, and O. Ronneberger, “Clinically applicable deep learning for diagnosis and referral in retinal disease,” Nat. Medicine (2018).
47. J. Wang, X. Yang, H. Cai, W. Tan, C. Jin, and L. Li, “Discrimination of Breast Cancer with Microcalcifications on Mammography by Deep Learning,” Sci. Reports 6 (2016).
48. R. Ha, P. Chang, J. Karcich, S. Mutasa, E. Pascual Van Sant, M. Z. Liu, and S. Jambawalikar, “Convolutional Neural Network Based Breast Cancer Risk Stratification Using a Mammographic Dataset,” Acad. Radiol. (2018).
19. R. Ha, P. Chang, S. Mutasa, J. Karcich, S. Goodman, E. Blum, K. Kalinsky, M. Z. Liu, and S. Jambawalikar, “Convolutional Neural Network Using a Breast MRI Tumor Dataset Can Predict Oncotype Dx Recurrence Score,” J. Magn. Reson. Imaging 0 (2018).
50. R. Ha, P. Chang, J. Karcich, S. Mutasa, E. P. Van Sant, E. Connolly, C. Chin, B. Taback, M. Z. Liu, and S. Jambawalikar, “Predicting Post Neoadjuvant Axillary Response Using a Novel Convolutional Neural Network Algorithm,” Annals Surg. Oncol. (2018).
51. R. Ha, P. Chang, J. Karcich, S. Mutasa, R. Fardanesh, R. T. Wynn, M. Z. Liu, and S. Jambawalikar, “Axillary Lymph Node Evaluation Utilizing Convolutional Neural Networks Using MRI Dataset,” J. Digit. Imaging (2018).
52. B. E. Bejnordi, M. Veta, P. J. Van Diest, B. Van Ginneken, N. Karssemeijer, G. Litjens, J. A. Van Der Laak, M. Hermsen, Q. F. Manson, M. Balkenhol, O. Geessink, N. Stathonikos, M. C. Van Dijk, P. Bult, F. Beca, A. H. Beck, D. Wang, A. Khosla, R. Gargeya, H. Irshad, A. Zhong, Q. Dou, Q. Li, H. Chen, H. J. Lin, P. A. Heng, C. HaB, E. Bruni, Q. Wong, U. Halici, M. A. Oner, R. Cetin-Atalay, M. Berseth, V. Khvatkov, A. Vylegzhanin, O. Kraus, M. Shaban, N. Rajpoot, R. Awan, K. Sirinukunwattana, T. Qaiser, Y. W. Tsang, D. Tellez, J. Annuscheit, P. Hufnagl, M. Valkonen, K. Kartasalo, L. Latonen, P. Ruusuvuori, K. Liimatainen, S. Albarqouni, B. Mungal, A. George, S. Demirci, N. Navab, S. Watanabe, S. Seno, Y. Takenaka, H. Matsuda, H. A. Phoulady, V. Kovalev, A. Kalinovsky, V. Liauchuk, G. Bueno, M. M. Fernandez-Carrobles, I. Serrano, O. Deniz, D. Racoceanu, and R. Venancio, “Diagnostic assessment of deep learning algorithms for detection of lymph node metastases in women with breast cancer,” JAMA—J. Am. Med. Assoc. 318, 2199-2210 (2017).
53. D. C. Ciresan, A. Giusti, L. M. Gambardella, and J. Schmidhuber, “Mitosis Detection in Breast Cancer Histology Images with Deep Neural Networks,” in Medical Image Computing and Computer-Assisted Intervention—MICCAI 2013, K. Mori, I. Sakuma, Y. Sato, C. Barillot, and N. Navab, eds. (Springer Berlin Heidelberg, Berlin, Heidelberg, 2013), pp. 411-418.
54. J. T. Springenberg, A. Dosovitskiy, T. Brox, and M. Riedmiller, “Striving for Simplicity: The All Convolutional Net,” arXiv [cs.LG] (2014).
55. V. Nair and G. E. Hinton, “Rectified Linear Units Improve Restricted Boltzmann Machines,” Proc. 27th Int. Conf. on Mach. Learn. pp. 807-814 (2010).
56. S. Ioffe and C. Szegedy, “Batch Normalization: Accelerating Deep Network Training by Reducing Internal Covariate Shift,” arXiv:1502.03167 pp. 1-11 (2015).
57. K. He, X. Zhang, S. Ren, and J. Sun, “Delving deep into rectifiers: Surpassing human-level performance on imagenet classification,” in Proceedings of the IEEE International Conference on Computer Vision, vol. 2015 Inter (2015), pp. 1026-1034.
58. D. P. Kingma and J. Ba, “Adam: A Method for Stochastic Optimization,” IEEE Signal Process. Lett. (2014).
59. Y. Bengio, N. Boulanger-Lewandowski, and R. Pascanu, “Advances in optimizing recurrent networks,” in ICASSP, IEEE International Conference on Acoustics, Speech and Signal Processing—Proceedings, (2013), pp. 8624-8628.
60. D. P. Mandic, “A generalized normalized gradient descent algorithm,” (2004).
61. J. Landercasper, E. Whitacre, A. C. Degnim, and M. Al-Hamadani, “Reasons for Re-Excision After Lumpectomy for Breast Cancer: Insight from the American Society of Breast Surgeons Mastery<sup>SM</sup> Database,” Annals Surg. Oncol. 21, 3185-3191 (2014).
62. J. F. Waljee, E. S. Hu, L. A. Newman, and A. K. Alderman, “Predictors of Breast Asymmetry after Breast-Conserving Operation for Breast Cancer,” J. Am. Coll. Surg. 206, 274-280 (2008).
63. K. Simiyoshi, T. Nohara, M. Iwamoto, S. Tanaka, K. Kimura, Y. Takahashi, Y. Kurisu, M. Tsuji, and N. Tanigawa, “Usefulness of intraoperative touch smear cytology in breast-conserving surgery,” Exp. Ther. Medicine 1, 641-645 (2010).
64. J. C. Cendán, D. Coco, and E. M. Copeland, “Accuracy of intraoperative frozen-section analysis of breast cancer lumpectomy-bed margins,” J. Am. Coll. Surg. 201, 194-198 (2005).
65. T. E. Doyle, R. E. Factor, C. L. Ellefson, K. M. Sorensen, B. J. Ambrose, J. B. Goodrich, V. P. Hart, S. C. Jensen, H. Patel, and L. A. Neumayer, “High-frequency ultrasound for intraoperative margin assessments in breast conservation surgery: a feasibility study,” BMC Cancer 11, 444 (2011).
66. S. Goldfeder, D. Davis, and J. Cullinan, “Breast Specimen Radiography. Can It Predict Margin Status of Excised Breast Carcinoma?” Acad. Radiol. 13, 1453-1459 (2006).
67. F. Schnabel, S. K. Boolbol, M. Gittleman, T. Karni, L. Tafra, S. Feldman, A. Police, N. B. Friedman, S. Karlan, D. Holmes, S. C. Willey, M. Carmon, K. Fernandez, S. Akbari, J. Harness, L. Guerra, T. Frazier, K. Lane, R. M. Simmons, A. Estabrook, and T. Allweis, “A randomized prospective study of lumpectomy margin assessment with use of marginprobe in patients with nonpalpable breast malignancies,” Annals Surg. Oncol. 21, 1589-1595 (2014).
68. Z. Burgansky-Eliash, G. Wollstein, T. Chu, J. D. Ramsey, C. Glymour, R. J. Noecker, H. Ishikawa, and J. S. Schuman, “Optical coherence tomography machine learning classifiers for glaucoma detection: a preliminary study.” Investig. ophthalmology & visual science 46, 4147-52 (2005).
69. R. J. Zawadzki, A. R. Fuller, D. F. Wiley, B. Hamann, S. S. Choi, and J. S. Werner, “Adaptation of a support vector machine algorithm for segmentation and visualization of retinal structures in volumetric optical coherence tomography data sets,” J. Biomed. Opt. 12, 041206 (2007).
70. A. Abdolmanafi, L. Duong, N. Dandah, and F. Cheriet, “Deep feature learning for automatic tissue classification of coronary artery using optical coherence tomography,” Biomed. Opt. Express 8, 1203 (2017).
71. G. Zahnd, A. Karanasos, Â. Gijs Van Soest, E. Regar, W. Niessen, F. Gijsen, T. Van Walsum, A. Karanasos, Â. E. Regar, G. Van Soest, and A. F. Gijsen, “Quantification of fibrous cap thickness in intracoronary optical coherence tomography with a contour segmentation method based on dynamic programming,” Int J CARS 10, 1383-1394 (2015).
72. A. Coates, A. Arbor, and A. Y. Ng, “An Analysis of Single-Layer Networks in Unsupervised Feature Learning,” Aistats 2011 pp. 215-223 (2011).
73. G. Marcus, “Deep Learning: A Critical Appraisal,” arXiv preprint arXiv:1801.00631 pp. 1-27 (2018).
74. Clough, K. B. et al. Oncoplastic techniques allow extensive resections for breast-conserving therapy of breast carcinomas. Ann. Surg. 237, 26-34 (2003).
75. Tartter, P. I. et al. Lumpectomy margins, reexcision, and local recurrence of breast cancer. Am. J. Surg. 179, 81-85 (2000).
76. Cendán, J. C., Coco, D. & Copeland, E. M. Accuracy of intraoperative frozen-section analysis of breast cancer lumpectomy-bed margins. J. Am. Coll. Surg. 201, 194-198 (2005).
77. Goldfeder, S., Davis, D. & Cullinan, J. Breast Specimen Radiography. Can It Predict Margin Status of Excised Breast Carcinoma? Acad. Radiol. 13, 1453-1459 (2006).
78. Schnabel, F. et al. A randomized prospective study of lumpectomy margin assessment with use of marginprobe in patients with nonpalpable breast malignancies. Ann. Surg. Oncol. 21, 1589-1595 (2014).
79. Ha, R. et al. Optical Coherence Tomography: A Novel Imaging Method for Post-lumpectomy Breast Margin Assessment—A Multi-reader Study. Acad. Radiol. (2017). doi:10.1016/j.acra.2017.09.018
80. Yao, X., Gan, Y., Marboe, C. C. & Hendon, C. P. Myocardial imaging using ultrahigh-resolution spectral domain optical coherence tomography. J. Biomed. Opt. 21, 061006 (2016).
81. Brady, A. P. Error and discrepancy in radiology: inevitable or avoidable? Insights Imaging 8, 171-182 (2017).
82. LeCun, Y. A., Bengio, Y. & Hinton, G. E. Deep learning. Nature 521, 436-444 (2015).
83. Krizhevsky, A., Sutskever, I. & Geoffrey E., H. ImageNet Classification with Deep Convolutional Neural Networks. Adv. Neural Inf. Process. Syst. 25 1-9 (2012). doi:10.1109/5.726791

1. A non-transitory computer-accessible medium having stored thereon computer-executable instructions for classifying at least one breast tissue of at least one patient, wherein, when a computer arrangement executes the instructions, the computer arrangement is configured to perform procedures comprising:
receiving at least one image of at least one internal portion of a breast of the at least one patient; and
automatically classifying the at least one breast tissue of the breast by applying at least one neural network to the at least one image.

2. The computer-accessible medium of claim 1, wherein the automatic classification includes a classification as to whether the at least one breast tissue is at least one of atypical ductal hyperplasia or ductal carcinoma.

3. The computer-accessible medium of claim 1, wherein the automatic classification includes a classification as to whether the at least one breast tissue is a cancerous tissue or a non-cancerous tissue.

4. The computer-accessible medium of claim 1, wherein the at least one image is a mammographic image.

5. The computer-accessible medium of claim 1, wherein the at least one image is an optical coherence tomography image.

6. The computer-accessible medium of claim 1, wherein the neural network is a convolutional neural network (CNN).

7. The computer-accessible medium of claim 6, wherein the CNN includes a plurality of layers.

8. The computer-accessible medium of claim 7, wherein the layers include (i) a plurality of residual layers, (ii) a plurality of inception layers, (iii) at least one fully connected layer, and (iv) at least one linear layer.

9. The computer-accessible medium of claim 8, wherein (i) the residual layers include at least four residual layers, (ii) the inception layers include at least four inception layers, (iii) the at least one fully connected layer includes at least sixteen neurons, and (iv) the at least one linear layer includes at least eight neurons.

10. The computer-accessible medium of claim 7, wherein the layers include (i) a plurality of combined convolutional and rectified linear unit (ReLu) layers, (ii) a plurality of partially strided convolutional layers, (iii) a plurality of ReLu layers, and (iv) a plurality of fully connected layer.

11. The computer-accessible medium of claim 10, wherein (i) the combined convolutional and ReLu layers include at least three combined convolutional and ReLu layers, (ii) the partially strided convolutional layers include at least three partially strided convolutional layers, (iii) the ReLu layers include at least three ReLu layers, and (iv) the fully connected layer includes at least 15 fully connected layers.

12. The computer-accessible medium of claim 1, wherein the computer arrangement is further configured to determine at least one score based on the at least one image using the at least one neural network.

13. The computer-accessible medium of claim 12, wherein the computer arrangement is configured to automatically classify the breast tissue based on the score.

14. The computer-accessible medium of claim 13, wherein the computer arrangement is configured to automatically classify the breast tissue based on the score being above 0.5.

15. The computer-accessible medium of claim 1, wherein the at least one image illustrates at least one excised breast tissue.

16. The computer-accessible medium of claim 1, wherein the computer arrangement is further configured to segment and resize the at least one image prior to classifying the breast tissue.

17. The computer-accessible medium of claim 1, wherein the computer arrangement is further configured to perform a batch normalization on the at least one image.

18. The computer-accessible medium of claim 17, wherein the computer arrangement is configured to perform the batch normalization so as to limit a drift of layer activations.

19. A method for classifying at least one breast tissue of at least one patient, comprising:
receiving at least one image of at least one internal portion of a breast of the at least one patient; and
using a computer arrangement, classifying the at least one breast tissue of the breast by applying at least one neural network to the at least one image.

20-36. (canceled)

37. A system for classifying at least one breast tissue of at least one patient, comprising:
a computer hardware arrangement configured to:
receive at least one image of at least one internal portion of a breast of the at least one patient; and
classify the at least one breast tissue of the breast by applying at least one neural network to the at least one image.

38.-54. (canceled)
